(19) United States
(12) Patent Application Publication
(10) Pub. No.: US 2016/0183789 A1
(43) Pub. Date: Jun. 30, 2016

(54) USER INITIATED AND FEEDBACK CONTROLLED SYSTEM FOR DETECTION OF BIOMOLECULES THROUGH THE EYE

(71) Applicant: HIGI SH LLC, Chicago, IL (US)

(72) Inventors: Shin-Yuan Yu, Overland Park, KS (US); Robin Taylor, Reading (GB); James Reynolds, Stroud (GB); David Erceg, South Pasadena, CA (US); Colin K. Hill, San Dimas, CA (US); John Collins, Arcadia, CA (US); Jeffrey Flammer, Scottsdale, AZ (US)

(21) Appl. No.: 14/984,715

(22) Filed: Dec. 30, 2015

Related U.S. Application Data
(60) Provisional application No. 62/098,806, filed on Dec. 31, 2014.

(51) Int. Cl. A61B 3/15; A61B 3/00; A61B 3/113

(52) U.S. Cl. CPC A61B 3/152; A61B 3/113; A61B 3/0025; A61B 3/0041

(57) ABSTRACT

A system for detecting biomolecules in a user's eye having a head and tracking system positioned a comfortable distance from the user that provides positioning cues for the user, an optical system for providing scans of the user's eye, and a controller that operates the head and tracking system and the optical system and perform a risk analysis based on data from the scans.

### CROSS-REFERENCE TO RELATED APPLICATIONS

This application claims priority to Provisional Application U.S. Ser. No. 62/098,806 filed on Dec. 31, 2014, which is herein incorporated by reference in its entirety.

### BACKGROUND OF THE INVENTION

This invention is directed to a system for detection of biomolecules through the eye and more particularly to a detection system that uses both an eye and face positioning system and an optical system.

Known in the art is that measured biomolecules can be detected in biological tissues in-vivo. Specifically, numerous studies have shown that detecting biomolecules present in parts of the eye may correlate to progression of diseases and, further, lead to early detection of disorders. Several of these have been shown to increase in concentration in the eye with age. Of these several are related to disease conditions and may be used to indicate the risk category that a person has achieved for a specific disease with age or that a person has a particular disease. Based on this published scientific evidence inventors in prior art have developed methods where an operator uses a piece of equipment to position the eye of a patient and then take readings of certain biomolecules using light activation but with the lens of the exciting and receiving device only a few centimeters from the eye and the head held still by a mechanical device. At least one of these inventions uses a system whereby by moving the lenses of the system relative to the eye they can slowly scan through the thickness of the eye from front to back. These systems lack sensitivity for some applications, require a person to operate the system and to make sure the patient is properly oriented so the light enters the eye correctly and require the patient to sit with head forcibly held still for more than several seconds. Therefore, a detection system is desired that addresses these deficiencies.

An objective of the present invention is to provide a biomolecule detection system that does not require close positioning of an eye to an exciting and receiving device.

Another objective of the present invention is to provide a biomolecule detection system that does not require the use of a mechanical device to hold a user's head still.

A still further objective of the present invention is to provide a molecule detection system that is capable of use by a patient without the assistance of an operator and/or technician.

These and other objectives will be apparent to one skilled in the art based on the following written description, drawings and claims.

### SUMMARY OF THE INVENTION

In the current invention a new and novel system that addresses many of the issues limiting the previous devices is developed where a user (member of the public) can sit comfortably and at a comfortable distance from the device lens and following instructions or using the eye movement to position an avatar or similar guides on a LCD touch screen or other input device, position the head and the eye and hold the eye in a defined spot without mechanical intervention holding the head. The system then activates a scanning system that uses a laser or LED of defined wavelength to penetrate the eye, autofocus in defined planes through the eye and excite the biomolecule of interest. The excited molecule emits light that is then detected by the same system of optics and returned to the device for processing. The novel optical system also allows scatter and reflection of the incoming light to be detected and used for real time alignment during a test. Software is used interactively through the LCD screen and the device firmware system to train the user on positioning, control the scanning, decide which scans can be used and save and process the data. This system is novel in that the user's eye can be over 30 cm away from the light source, the system is user activated and controlled via interactive software, avatar gamifications software and touch screen images and there is thus an eye tracking system to position the eye so the user does not have to have the head restrained to hold a certain position. To achieve a compact design, physical dimensions of this system can also be reduced by utilizing mirror(s) in between the light source and optical lens. Results from measurements taken by the device are presented in the form of numbers and charts with color depicting the risk category the person is in for developing the disease of interest. The light emitting and scanning system is designed to complete more than 20 scans in less than 1.5 seconds thus giving enough data to form a reliable average value. The user therefore once comfortably sitting and activating the system can complete a test of the eye in only a few to several seconds.

### BRIEF DESCRIPTION OF THE DRAWINGS

FIG. 1 is a block diagram of a biomolecule detection system;

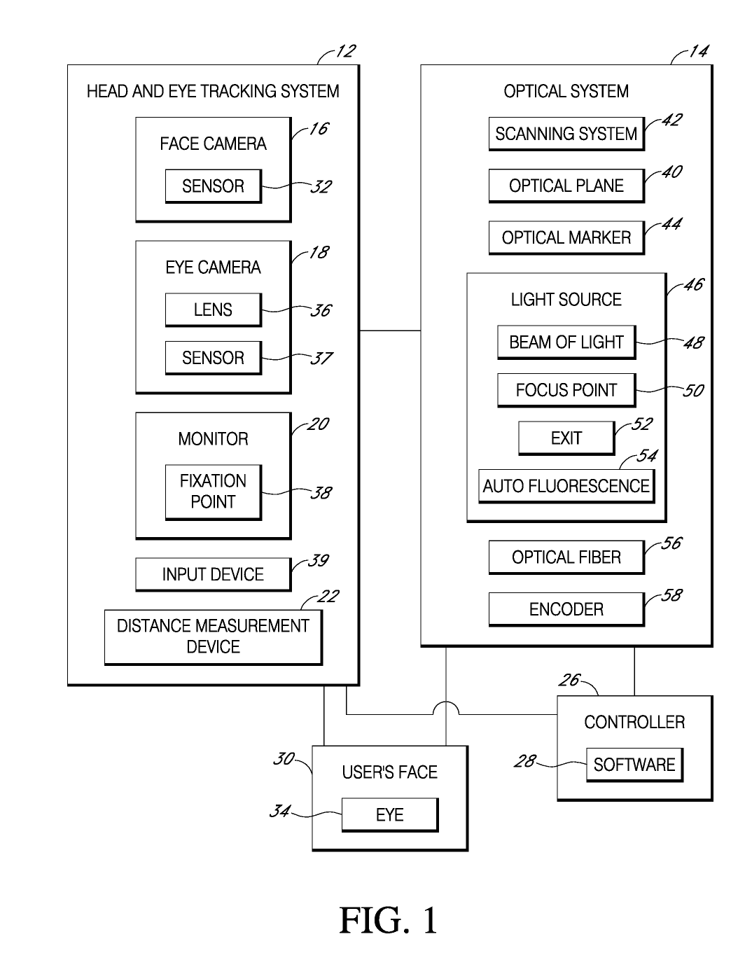

FIG. 2 is a flow diagram of a biomolecule detection system; and

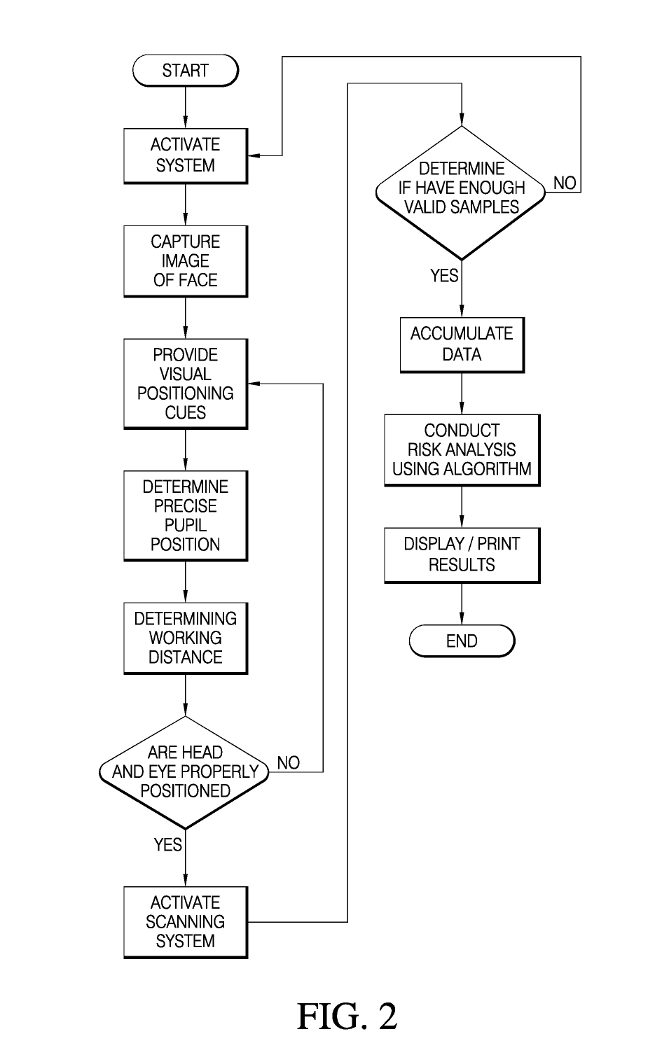

FIG. 3 is a perspective view of a biomolecule detection system.

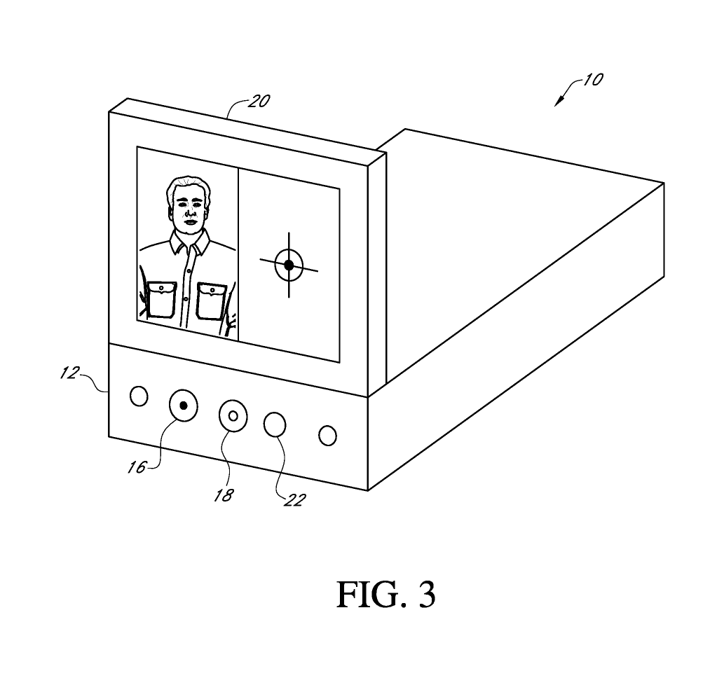

### DETAILED DESCRIPTION OF THE PREFERRED EMBODIMENTS

Referring to the Figures, a system for detection of biomolecules 10 through the eye includes a head and eye tracking system 12 and an optical system 14. The head and eye tracking system includes a face camera 16, an eye camera 18, a monitor 20, and a distance measurement device 22 that are connected to a controller 26 operated by software 28.

The face camera 16 is used to capture an image of a user's face 30 to provide information to the controller 26 for facial recognition and to provide visual positioning cues for the user. In one example, an expected range of resolution is 0.3-10 Mega-pixel (MP) and preferably is 1.5 to 5 MP. The face camera 16 is equipped with a CCD or CMOS sensor 32 and can be a color or monochrome camera 16. An expected useful frame rate in one example is 30 to 120 frames per second (fps) and preferably 60 fps.

The eye camera 18 captures a close-up image of the eye 34 of a user to provide information to the controller 26 of precise pupil position used to control the optical scanning system 14. An expected range of resolution is 0.3 to 10 MP and preferably 1.0 to 3.5 MP. The eye camera 18, in one example, preferably is equipped with a fixed or variable zoom-in lens 36 having a focal length of 8.5 to 120 mm and preferably a variable zoom-in lens 36 with a focal length of 9 to 90 mm. The eye camera 18 may be color or monochrome broadband which covers near infrared (near-IR, e.g., 850 nm) in wavelength spectrum. The working distance defined as the distance between the surface of the eye and the emitting and receiving lens 36 is determined by the eye camera 18 with the distance measurement device 22 and the expected range in one example is between 300 to 600 mm. The eye camera 18 is equipped with a CCD or CMOS sensor 37 with a frame rate in one example of 30 to 300 fps. In one embodiment, the single camera is used for both the face camera 16 and the eye camera 18.

The monitor 20 may be part of the system 10 or an LCD screen for a health kiosk, a health workstation or a laptop screen connected to the system 10 is used as a monitor 20. The monitor 20 allows for critical interaction between the monitor 20 and a user. The monitor renders visual feedback for self-positioning, provides a fixation point 38 as a target for the user's gaze, directs the user to position their head and align the eye for measurement purposes, accepts user manual inputs when a touch-screen is utilized and can be used to present the user with an interactive self-assisting avatar that helps the user move into the correct position, similar to a video game or the like. Any other input device 39 is used in place of a touch screen to accept manual inputs.

Since the focal length of the optical system 14 is fixed at the working distance, one constraint is to have precise measurement of the distance between the users head and the optical plane 40 of the system. The distance measurement device 22 may be based on small ultrasonic detectors, time of flight measurements using low light infrared emitters, a form of Doppler radar, or feedback systems using scattered light from the incoming laser or LED light. Scattered and reflected light permits tracking of the surface of the cornea and the retina. The measurement device 22 precisely measures the distance between markers, such as the bridge of the nose on a user's head or the head itself and the reference plane 40 of the system, and provides information about the head orientation on the transverse plane. The distance measurement device 22 in one example has a resolution of 1 to 5 mm and an expected applicable range of 20 to 5,000 mm. The expected power supply for the distance measurement device 22 in one example is 5V with a quiescent current less than 2 mA. A sample rate in one example is between 5 and 1 kHz and preferably is about 10 Hz.

Accurate eye distance measurement will also enhance a self-administered test for standard eye exams. For example, reading issues at younger ages are not evaluated properly and can cause educational deficiencies. Eye exams are added medical coverages (not included in standard med-plan). This can lead to ADD/ADHD, or any learning disability. Public kiosk self-administered eye testing can help children at a very early age.

Also, accurate eye video photo enhancement algorithms will allow Pharmaceutical companies to evaluate drugs using a public kiosk self-test environment testing. Items like: Red eye, allergies, itchy eyes, dry eyes, medicine that will allow minor adjustment of the eye lens (without glasses or contact lenses); pupil deformities and eye redness can detect various medical conditions related to diabetes; and using IR base frequency sensors for eye illumination with filtering algorithms can help doctors see many issues with the surface of the eye that cannot be seen with standard eye equipment.

Once the head and eye are located and within the desired location the system 10 uses an optical system 14 that preferably is based on detecting reflections from the corneal surface to allow the software 28 to trigger the scanning system 42 of the biomolecule detection system 10. Purkinje reflections (i.e. P1, P3, and P4) can be used to provide the system 10 additional information about alignment and relative orientation between the lens and optical measuring unit. An optical marker 44 creates corneal reflections for iris detection with four units at distinct locations. The optical marker 44 also triggers the bright-eye effect for pupil detection with a unit on the optical axis of the eye camera 18. In one embodiment, the optical marker 44 is an infrared LED that can illuminate the user under low lighting conditions when near-infrared cameras are used for the facial and pupil detection. The output flux in one example for the optical marker 44 preferably is between 10 and 100 lm (lumens). Alternatively, an infrared camera and sensor used in combination with facial recognition software is used for detection. Also a blue laser may be used.

In operation a user receives positioning feedback from the system 10. Thus, the user has to be a reasonable reading distance from the monitor screen 20 in order to perceive visual feedback with assistance from the on-device sensors 22 while the tested eye is also fixating at a target 38. Preferably, in one example, the user's eye is 400 mm plus or minus 5 mm from the monitor screen 20 to facilitate the test for users of all ages. While preferred, tests may be conducted in another example at a distance as little as 20 cm and as great as 60 cm for some embodiments of the system 10. Still, any distance greater than 15 cm will work.

Facial detection is performed by the controller 26 using inputs detected by the face camera 16. The distance between the head and the monitor screen can be estimated by the controller 26 using a captured image of the face and further refined with measurements taken by the distance measurement device 22 or digital distance measurers. In addition, by applying two digital distance measurers the system 10 also allows head positioning in the lateral direction and transversal rotation for head positioning with precision. Based upon sensed information, the software 28 will prompt the user to adjust the head position until the head is properly positioned.

The controller 26 will also provide a visual fixation target 38 display on the monitor 20 overlapped with a close-up reflection of the eye 34. Through the eye camera 18 and the software 28 the position of the eye 34 is continuously monitored to determine the center of the pupil in real-time or sequentially information about an area of the eye/pupil is built up based on natural movements of the eye including saccades.

The user is prompted by the controller 26 to fixate their gaze at the target 38 and rotate/move their head in order to coincide the fixation point with the center of the pupil. A measurement by the optically driven biomolecule detection device 10 is performed whenever this occurs. Thus, the optically driven biomolecule detection device 10 system takes a reading or multiple readings per second as controlled by the software 28.

The optical system 14 has a light source 46 that will direct a beam of light 48 to focus at a point 50 (i.e., target, object, eye) that is, in one embodiment of the invention 500 mm away from the exit 52 of the optics and detects the returned auto fluorescence 54 from biomolecules of interest in the eye. In one embodiment, of the detection system in the eye will cover a range of 32 mm in the eye.

The light source 46 has a wavelength typically between 400 and 520 nm, in order to excite fluorescence from molecules within the target 50. Scattered and reflected light will have the same wavelength as the source, whereas emitted (fluorescent) light will have a longer wavelength (typically 20-50 nm longer than the excitation wavelength). The optimum wavelength is determined by a number of parameters, such as eye safety, quantum yield, detector responsivity, component cost, and the like.

The optical system 14 follows the principle of confocal microscopy, wherein light from a small (pinhole) source is focused onto a small region within the object. The requirements for the confocal system here are unusual in a number of respects. First, the target object is much further from the optical system than the image, making the magnification less than 1. Second, the source rather than the lenses are scanned (to achieve a reasonable NA the lenses are too large to make scanning practicable). Third, the system needs to be simple and reasonably small, both to keep costs down and fit into the available space. Being a fluorescent system, the optics must also be well-corrected for lateral chromatic aberration, as well as producing a diffraction-limited spot and this must be maintained over the source scan range.

For these reasons, the optical system needs to be closer to a telescope than a microscope. A particular class of telescope, catadioptric dialytes (catadioptric: using both reflective and refractive elements; dialyte: chromatic correction performed by widely-spaced elements), forms a good starting point for a suitable design. Telescopes in this class have good monochromatic optical performance combined with intrinsically low lateral chromatic aberration using few optical components, and with folded optics have a short overall length. The simplest of these is the Hamiltonian telescope, which uses only two elements: two lenses, one of which has a reflective coating.

Hamilton's telescope used two singlet lenses. Here these have been replaced by doublets, which allow apochromatic correction. The design is not especially sensitive to glass properties, which means that common low-cost optical glasses can be used. The optical system used in a confocal meter is modified to maintain the apochromatic correction and diffraction-limited performance whilst reducing the focal length and ensuring that this performance is maintained across the scan range. It has also been optimized for tolerances, ensuring that the required performance is achievable without excessive alignment and manufacturing accuracy requirements. Due to the geometrical arrangement light scattered or emitted from other regions of the target, the object is either not detected or detected at vastly reduced intensity.

Preferably, the source and detector pinholes are formed by a single optical fiber 56. This configuration means that the whole system is self-aligning and does not require accurate alignment of the various optical components. However, this puts the additional requirement on the optical system 14 that it must be highly achromatic; the focal shift between the source and emitted wavelengths must be less than the full width half max (FWHM) of the focal spot. Excitation wavelengths in one example of between 400 and 532 nm. In yet another example, detection wavelengths are 20 nm to 120 nm greater than excitation wavelengths. Detection wavelengths in another example in the range of 515 to 580 nm are preferred. Small lasers or small band LED light sources may be used as well as any source of coherent or incoherent light.

In order to provide depth resolution within the target 50, the focus is scanned in the z direction. Rather than scanning the lens (as in conventional confocal microscopy) the fiber 56 is scanned, this is much smaller and lighter than the lens and allows much more rapid scanning. If a meaningful relation between depth and signal is to be established, the target 50 must be essentially static over the scan. If the target 50 is the human eye 34, low speed scanning (<˜20 scans/s) requires that the position of the head and eye is constrained for it to be static over the scan period. With more rapid scanning head motion need not be constrained, since the eye position will remain essentially constant over the scan period. Scanning the fiber 56 has the additional advantage that it does not limit the range at which the head can be placed: as the working distance increases so must the lens diameter, and moving a large and heavy lens rapidly is impracticable.

The focal position is a function of the fiber position: this is measured using an encoder 58 and the range of the focal spot calculated from the fiber position. Data acquisition is triggered from the encoder 58 as described by U.S. Pat. No. 8,552,892 incorporated by reference in its entirety herein (the '892 patent). The optical system 14 coupled with the facial and eye tracking system 12 gives the biomolecule detection device a resolution accuracy at the focal point in the order of about 0.25 mm. This level of position accuracy allows the biomolecule detection device to be precisely driven by software 28 to record between 1 to 100 scans through the eye 34 by precisely incremented autofocus in a short period of time.

Axial resolution within the eye 34 is limited by the FWHM of the focal spot: in one example the axial resolution is ˜0.25 mm. Axial resolution better than 0.4 mm is preferred, in order to provide meaningful information about the distribution of biomolecules within the eye 34. Preferably, in one example, the axial resolution is between 220 and 550 microns and the lateral resolution is between 5 and 14 micrometers.

The biomolecule detection device in one example has an optical working range of 30 to 60 cm and preferably is 40 cm from the eye surface to the final, output lens. In another example the working distance is 500 mm and the scan range 32 mm. This is to allow the head to be placed in a comfortable position and a scan to be performed through the anterior chamber and crystalline lens. Other distances and ranges are of course possible, working distances in the range 300-600 mm and scan ranges up to 60 mm are achievable whilst meeting the axial resolution requirement.

The choice of wavelengths and use of a single mode fiber 56 as source and detector pinhole allow standard fiber optic components to be used. In particular, fluorescent emission may be separated from outgoing and scattered light by a wavelength division multiplexer 60 (green/blue splitter combiner), and the outgoing and returning scattered light separated by a fiber optic splitter 62 operating at the appropriate wavelength. Both components have high isolation and crosstalk can be kept below −50 dB. Noise in the system is largely due to shot noise from this crosstalk: the low crosstalk allows noise to have low amplitude and allows detection of low concentrations of fluorescent and scattering biomolecules. The splitter 62 operates at source wavelength.

In the case of the single wavelength splitter 62, optimum signal to noise ratio in one example is obtained when the splitting ratio is 66:33. Alternatively, 70:30 splitters 62 are readily available commercially and this small change in ratio makes little difference to the noise performance. Although not optimal, the system will operate with other splitting ratios, however it is preferred that more of the returned light is coupled into the detector than into the laser. If required, the laser may also be modulated in order to reduce 1/f noise.

To overcome the crosstalk inherent in the optical system, an optical time delay is introduced via a long optical path length and the light source is modulated. The processing of the returned signal is therefore displaced in time when compared to the outgoing signal so optical crosstalk does not affect the returned signal since the light source is off whilst the returned signal is being measured. The optical time delay depends on the length of the light source modulation pulse and the amount of time for any circuitry to recover from any possible overload due to optical crosstalk. To minimize the effects of a possible crosstalk overload of the electronics, one or more of the following methods can be used: First, the optical detector transimpedance amplifier is clamped to reduce its transimpedance or otherwise reduce the sensitivity of the first amplifier connected to the optical detector. Second, the output of the first or subsequent amplifiers is switched so that any disturbance due to crosstalk and/or clamping is not passed to subsequent signal processing stages.

Demodulation of the signal after initial processing/amplification is by a sample and hold and/or low pass filter which is gated at a fixed time delay after the light source pulse to take account of the optical delay. For example, if the light source pulse was optically delayed by 1 μs, then the sample and hold gating would be triggered approximately 1 μs after the light source has been turned on. The duration of the gating would be the same width as the light source pulse or less. The exact timing of the delay would depend on other electronic delays and bandwidths in the system so would not necessarily be exactly the same as the optical delay. Similarly, the sampling pulse could be reduced width compared to the light source modulation width.

Low pass filtering is used after demodulation to smooth out the measured signal and remove the modulation frequency. The pulse repetition rate of the light source would normally be as fast as possible within the optical time delay and pulse widths used to ensure that the light source is not turned on before sampling of the previous, delayed pulse has been completed.

Since crosstalk forms a significant proportion of the light reaching the detectors 64, which is at a constant level, detectors 64 are preferably blue-enhanced silicon photodiodes. Detectors 64 optimized for low light levels, e.g. APDs or PMTs, may be used, but offer no significant advantage in the presence of significant levels of crosstalk. The detectors 64 are pigtailed to the optical fiber and are connected to a dedicated amplifier 66. The amplifier 66 includes one or more stages and typically the first is a trans-impedance amplifier. Each stage has an adjustable input bias allowing for the compensation of cross talk to maximize the dynamic range of the amplifier 66.

For receptions of optical signals, the on-axis measurement that is described before is employed: The reference and fluorescence channels are identical even though the signal levels will be different. The gain and offset adjustment ranges allow identical channels.

The output signal is filtered with a 4 pole Bessel filter to limit bandwidth to 3 kHz and minimize pulse distortion with dynamic signals.

The signal received by the detector 64 is processed and is analyzed by the software 28 and the result displayed as a number between 1 and 100, one being a low and 100 being a high value of the biomolecule being measured. The software 28 then displays the result to the user as a risk chart or picture where green is normal, yellow is low risk and orange is significant risk of the disease process being tested.

An IC1 is configured as a transimpedance amplifier 68 with high transimpedance, 50 MΩ, so giving 50 mV/nA of photocurrent. Typical photodiodes 70 are 0.3 A/W at the wavelengths used. The photodiode 70 and transimpedance amplifier 68 have a screening to minimize noise pickup. Multiple feedback resistors minimize the effects of stray capacitance across the resistors to maximise bandwidth. A capacitive T network in the feedback allows a finely adjustable low value of compensation capacitance without using very low value capacitors. Bandwidth is around 12.5 kHz so it can be restricted to 3 kHz with the final filter. The opamp is chosen for low offset and low offset drift (0.4 μV/° C.) as there is potentially a very high DC gain required due to the low signal levels on the fluorescence channel.

The photodiode 70 can be reverse biased or zero biased. Reverse biasing is preferred for speed but zero biasing is preferred if there is significant photodiode 70 leakage current (and hence leakage drift with temperature). The opamp is biased at 2.5V. The photodiode 70 can be biased to 2.5V or 5V. A 16 bit DAC (IC4) allows the offset of the two subsequent gain stages (IC9) to be adjusted independently. Depending on the gain setting of the two stages this gives a coarse and fine control. The DAC can use the same 2.5V reference (IC5) as the transimpedance amplifier/photodiode or it can use a separate 3V reference (IC6). The 3V reference would only be used if there is insufficient crosstalk and too high an opamp offset to adjust the output voltage within the required range. The references are ultra-low noise high stability ones—typically 1 ppm/° C.

Two gain stages (IC10A/B) allow a gain of up to 256 per stage giving a total gain of 65,536 although the maximum likely gain used will be around 10,000 and most likely less. The gain is controlled by a dual digital potentiometer (IC8) with 256 steps per arm controlled in “independent mode”. This allows a theoretical 65,526 gain settings per stage although some of those will be duplicates and others will be such small differences from the nearest step that they are of no practical use. It does, however, mean that almost any gain can be set between minimum and maximum, independently for each stage.

The output signal is filtered with a 4 pole Bessel filter to limit bandwidth to 3 kHz and minimize pulse distortion with dynamic signals.

The laser can be powered directly from the LDA PCB if the current is not too high or optionally via an alternative power supply. The laser power is controlled by a 12 bit DAC (IC11) with EEPROM storage and the laser can be turned on/off with a logical signal from the MPB.

Data is accumulated from scans validated by the controller. The controller determines if enough scans are available for a sample and, if there are, the controller conducts a risk analysis. In addition to data of identified biomolecules from the scans, the controller may use other biometric information input by a user to complete the risk analysis. The inputted information may include other biometric parameters, such as body composition, weight, blood pressure, gender, and the like, that are clinically associated with diabetes to improve the risk analysis.

Once completed, the risk analysis is displayed on the monitor and/or printed on a printing device.

Thus, a biomolecule detection system has been disclosed that at the very least meets all the stated objectives.

What is claimed:

1. A system for detecting biomolecules, comprising:
a head and eye tracking system connected to a controller having software;
an optical system connected to the controller;
wherein the software is configured to provide positioning cues through the head and eye tracking system to position a user, activate the optical system when user is in a desired position, and conduct a risk analysis based upon scans taken by the optical system.

2. The system of claim 1 wherein the head and eye tracking system includes a face camera, an eye camera, a monitor, and a distance measurement device.

3. The system of claim 1 wherein the optical system has a scanning system and a light source.

4. The system of claim 1 wherein a facial camera is configured to capture an image of a user's face that is used to provide the positional cue for positioning the head.

5. The system of claim 4 wherein the positional cue is an avatar displayed on the monitor.

6. The system of claim 2 wherein the eye camera and the controller are configured to provide positioning cues for the user's pupil.

7. The system of claim 1 wherein the controller determines a focal point in a user's eye.

8. The system of claim 1 wherein the optical system is confocal.

9. The system of claim 1 wherein the optical system is a Hamiltonian lens system.

10. The system of claim 1 wherein the desired position is greater than 15 cm.

11. A method of detecting biomolecules, comprising the steps of:
providing positional cues through a head and eye tracking system to position a user's head and eye in a desired position;
determining, using a controller, a working distance between;
activating an optical system with the controller when the user's head and eye are in the desired position; and
conducting a risk analysis based upon identified biomolecules in scans of the user's eye using an algorithm in the controller.

12. The method of claim 11 further comprising the step of capturing an image of the user's face.

13. The method of claim 11 where the step of providing positional cues includes a display of an avatar on a monitor.

14. The method of claim 11 further comprising the step of determining, with the controller, the precise position of the user's pupil.

15. The method of claim 11 further comprising the step of validating the scans with the controller.

16. The method of claim 11 further comprising the step of determining if enough scans have been taken to conduct a risk analysis using the controller.

17. The method of claim 11 further comprising the step of displaying results of the risk analysis on a monitor using the controller.
